(19) United States
(12) Patent Application Publication
(10) Pub. No.: US 2026/0013904 A1
(43) Pub. Date: Jan. 15, 2026

(54) FRACTURE REDUCTION ROBOT HAVING WIRELESS DRIVE PART FORMED THEREIN

(71) Applicant: AIRS INC., Daegu (KR)

(72) Inventor: Sanghyun JOUNG, Daegu (KR)

(21) Appl. No.: 18/992,894

(22) PCT Filed: Jun. 30, 2023
(86) PCT No.: PCT/KR2023/009199

§ 371 Date: Oct. 3, 2025

(30) Foreign Application Priority Data
Jul. 14, 2022 (KR) 10-2022-0086731

(51) Int. Cl. A61B 17/66; A61B 17/00; A61B 34/35; B25J 13/00; B25J 19/00

(52) U.S. Cl. CPC A61B 17/66; A61B 34/35; B25J 13/006; B25J 19/0025; A61B 2017/00221; A61B 2017/00477

(57) ABSTRACT

Provided is a fracture reduction robot including a fracture reduction part having a plurality of ring frames for surrounding a fractured area of a patient and at least one or more struts located between the ring frames to adjust a distance between the ring frames and a wireless driving part connected to any one of the ring frames to transmit power for adjusting the distance to the struts, whereby during fracture reduction surgery, interferences with electric wires can be avoided.

### TECHNICAL FIELD

The present invention relates to a fracture reduction robot having a wireless driving part, more specifically to a fracture reduction robot having a wireless driving part, the driving part having a power source part and a wireless communication part and provided with an interface, so that electric wires causing interferences during fracture reduction surgery can be removed.

### BACKGROUND ART

In the case of the osteosynthesis for a patient with femur fracture or tibia fracture, a surgery assistance tool such as an external fixation device is fixed to a fractured bone, and then, the fractured bone is gradually set through repeated X-ray imaging so that the bone fracture is reduced.

In such a process, the surgery is performed by a surgeon who is positioned close to a C-arm X-ray machine, which undesirably causes the surgeon to be excessively exposed to radiation.

Further, after the fractured bone of the patient has been pulled by the surgeon, the pulled position of the fractured bone has to be maintained by the surgeon for a long period of time, which applies great physical strain on the surgeon. Therefore, a large number of surgeons are required to perform the needed physical labor for the osteosynthesis, which causes surgical manpower to be inefficiently managed.

A fracture table for the traction has been put on the market, but the fixation and traction of the fractured bone of an arm or a leg are difficult in adjusting accurate positions of fragments. Further, it is impossible to measure a force required for the fragment traction, which results in excessive traction.

Moreover, attempts using a surgical navigation system and a serial industrial robot arm have been made to solve the above-mentioned problems, but in this case, the payload of the robot is substantially large. As a result, to satisfy the payload of the robot, the robot becomes bulky, which fails to meet the design requirements needed in a surgical room.

According to Korean Patent No. 10-1735481 entitled ‘External fixation device having detachable actuator’, there is disclosed an external fixation device that allows detachable actuators to be easily mounted on variable legs so that it easily recognizes pre-states of the variable legs and thus performs the correction of the fractured or deformed bone successfully. However, components for pulling and reducing the bone increase, and if the external fixation device is fixed to the bone, electric wires for applying electricity to driving members for varying the lengths of the respective variable legs and the detachable actuators may extend, so that undesirably, the electric wires may come into contact with the bone to be reduced.

Therefore, there is a need to develop a fracture reduction robot having a wireless driving part so that during fracture reduction surgery, no interference with electric wires happens since the fracture reduction robot is provided with the wireless driving part from which the electric wires are removed.

### DISCLOSURE

### Technical Problem

Accordingly, the present invention has been made in view of the above-mentioned problems occurring in the related art, and it is an object of the present invention to provide a fracture reduction robot having a wireless driving part that is capable of allowing a battery and driving motors to be inserted into the wireless driving part, so that electric wires causing interferences during fracture reduction surgery can be removed.

### Technical Solution

To accomplish the above-mentioned objects, there is provided a fracture reduction robot according to an embodiment of the present invention including: a fracture reduction part having a plurality of ring frames for surrounding a fractured area of a patient and at least one or more struts located between the ring frames to adjust a distance between the ring frames; and a wireless driving part connected to any one of the ring frames to transmit power for adjusting the distance to the struts.

Further, the fracture reduction part may further include top connectors connected to one end of the struts and bottom connectors for connecting the other end of the struts to the driving part.

Furthermore, each strut may include: a strut body variable in length and having a sight gauge located thereon in a longitudinal direction thereof in such a way as to be transparent in the interior thereof and have graduations thereon and a measurer moving in the interior of the sight gauge according to the variation in length thereof to represent the length of the strut; a through hole formed on one end of the strut body in such a way as to be fastened to the corresponding top connector; and a power transmission member located on the other end of the strut body in such a way as to be connected to the corresponding bottom connector to receive the power for varying the length of the strut body from the driving part.

Moreover, each top connector may include: fixing pins passing through one end of the strut bodies in such a way as to allow the struts to be fixed thereto; escape prevention members coupled to the fixing pins in such a way as to prevent the struts fixed thereto from escaping therefrom; and rotational pins passing through one side peripheries of the fixing pins from the outside thereof in such a way as to adjust the angles of the fixing pins, whereby the struts are variable in angle around the rotational pins of the top connector.

Besides, the driving part may include: a driving part housing through which the patient's arm or leg passes; a controller located inside the driving part housing to provide the power to the struts; and driving part couplers located on one surface of the driving part housing and connected to the power transmission members through the bottom connectors in such a way as to transmit the power to the struts to adjust the lengths of the strut bodies.

In addition, each bottom connector may include first connection holes facing the corresponding struts and having diameters equal to the diameters of the power transmission members of the struts and second connection holes facing the corresponding driving part couplers and having diameters equal to the diameters of the driving part couplers, whereby the bottom connectors may connect the power transmission members and the driving part couplers to each other.

Further, each power transmission member may include: a pipe thread for coupling the corresponding strut to the corresponding bottom connector; a key groove engaged with the corresponding driving part coupler; and a ball joint having a pin passing therethrough from the outside thereof in such a way as to rotate around the pin.

Furthermore, the controller may include: a main controller for controlling the operation of the fracture reduction part; driving motors located to correspond to the struts in such a way as to vary the lengths and directions of the struts under the control of the main controller; and a power source part provided in the form of a battery in such a way as to apply a power source to the main controller and the driving motors.

Additionally, each driving part coupler may include clutch teeth engaged with the key groove and an elastic member for pushing the clutch teeth toward the key groove, and the clutch teeth may be engaged with the key groove through the operation of the controller.

A driving part of a fracture reduction robot according to another embodiment of the present invention may further include a cooler for lowering an amount of heat generated from the controller and mesh screens located on the side peripheral surfaces of the driving part where the driving motors are located in such a way as to allow the heat generated from the driving motors to be emitted to the outside, whereby cold air is transmitted directly or indirectly to the driving motors by means of the mesh screens.

A fracture reduction part of a fracture reduction robot according to yet another embodiment of the present invention may further include auxiliary connectors for surrounding the ring frame close to the driving part and the driving part in such a way as to fix the driving part to the ring frame.

### Advantageous Effects

According to the present invention, the fracture reduction robot is provided with the wireless driving part into which the battery and the driving motors are inserted, so that during the fracture reduction surgery, the fracture reduction robot can have no interference with electric wires.

### BRIEF DESCRIPTION OF DRAWINGS

FIG. 1 is a perspective view showing a fracture reduction robot having a wireless driving part according to an embodiment of the present invention.

FIG. 2A is an exploded perspective view showing a fracture reduction part and a driving part of FIG. 1, and FIG. 2B is a perspective view showing a state in which the fracture reduction part and the driving part are coupled to each other.

FIG. 3 is a side view showing a state in which the fracture reduction robot of FIG. 1 is fixed to a fractured area.

FIG. 4 is a perspective view showing the fracture reduction part of FIG. 2A.

FIG. 5 is a perspective view showing a state where top connectors of FIG. 4 are connected to a ring frame.

FIG. 6 is an exploded perspective view showing the top connectors and struts of FIG. 4.

FIG. 7 is a perspective view showing bottom connectors of FIG. 4.

FIG. 8 is an exploded perspective view showing the bottom connector to which the ring frame is connected, struts, and the driving part of FIG. 4.

FIG. 9 is a plan view showing the bottom connector of FIG. 7.

FIG. 10 is a perspective view showing the strut of FIG. 4.

FIG. 11 is a sectional view showing a state in which the strut of FIG. 10 is connected to the bottom connector.

FIG. 12 is a perspective view showing the driving part of FIG. 2A.

FIG. 13 is a perspective view showing a controller of the driving part of FIG. 12.

FIG. 14 is a perspective view showing a driving part coupler of FIG. 2A and a power transmission member of the strut.

FIG. 15 is a perspective view showing clutch teeth of the driving part coupler of FIG. 14.

FIGS. 16A to 16C are sectional views showing a state in which the driving part coupler of FIG. 14 is connected to the power transmission member of the strut.

FIG. 17 is a perspective view showing a fracture reduction robot having a wireless driving part according to another embodiment of the present invention.

FIG. 18 is an exploded perspective view showing the driving part and a cooler of FIG. 17.

FIG. 19 is a perspective view showing a fracture reduction robot having a wireless driving part according to yet another embodiment of the present invention.

FIG. 20 is a perspective view showing an auxiliary connector of FIG. 19.

FIG. 21 is a perspective view showing a state where the auxiliary connector of FIG. 20 is open.

FIGS. 22A to 22D are exemplary views showing a state in which the auxiliary connector of FIG. 19 is connected to the ring frame and the driving part.

### BEST MODE FOR INVENTION

Hereinafter, specific exemplary embodiments of the present invention are illustrated in the drawings and described in detail in the detailed description. However, this does not limit the invention within specific embodiments and it should be understood that the invention covers all the modifications, equivalents, and replacements within the idea and technical scope of the invention.

Terms, such as the first, the second, and the like, may be used to describe various elements, but the elements should not be restricted by the terms. The terms are used to only distinguish one element from the other element.

In the description, the same reference numerals will be used to describe the same components.

An expression referencing a singular value additionally refers to a corresponding expression of the plural number, unless explicitly limited otherwise by the context. In this application, terms, such as “comprise”, “include”, or “have”, are intended to designate those characteristics, numbers, steps, operations, elements, or parts which are described in the specification, or any combination of them that exist, and it should be understood that they do not preclude the possibility of the existence or possible addition of one or more additional characteristics, numbers, steps, operations, elements, or parts, or combinations thereof.

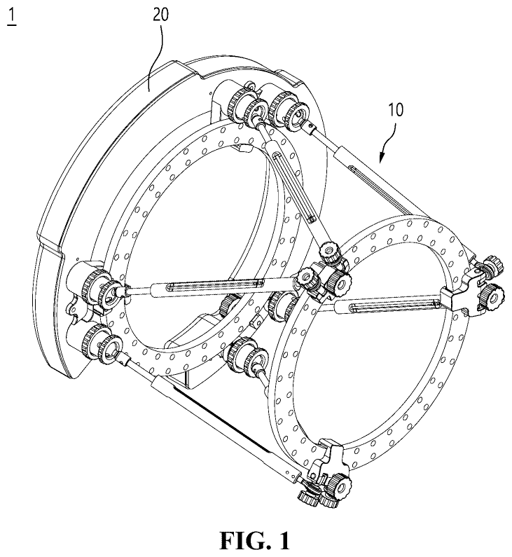

Hereinafter, an explanation of a fracture reduction robot having a wireless driving part according to an embodiment of the present invention will be given in detail with reference to FIGS. 1 to 22D.

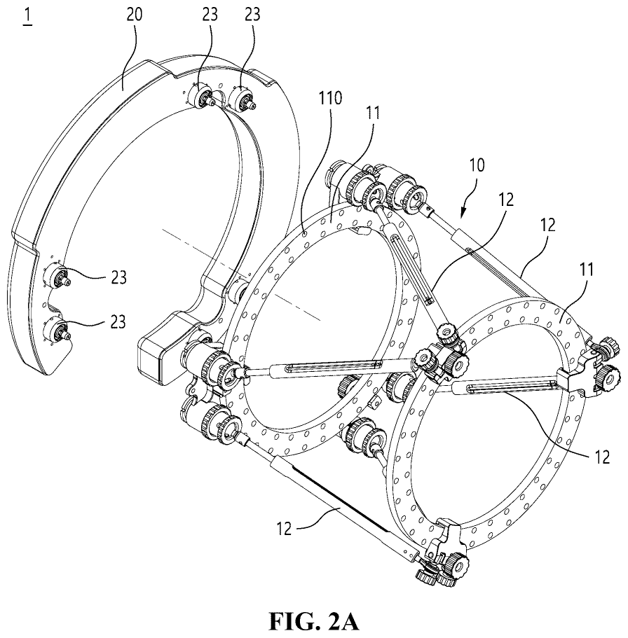

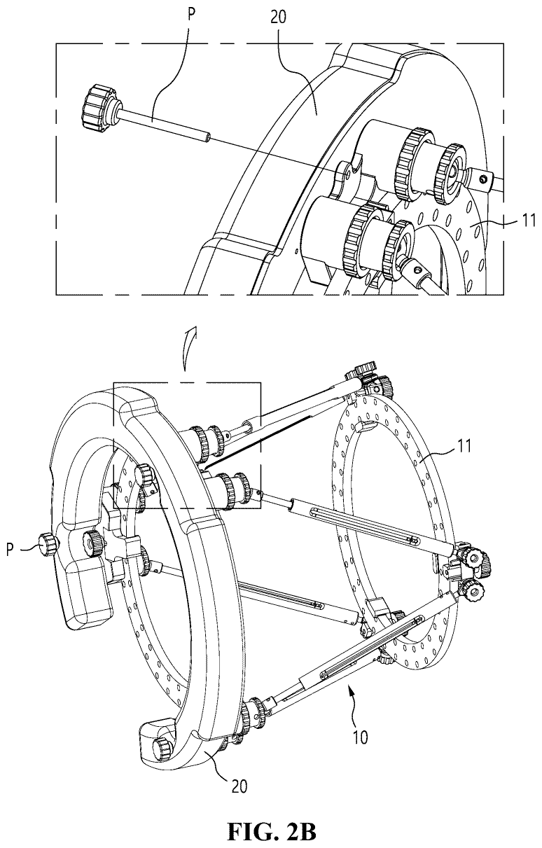

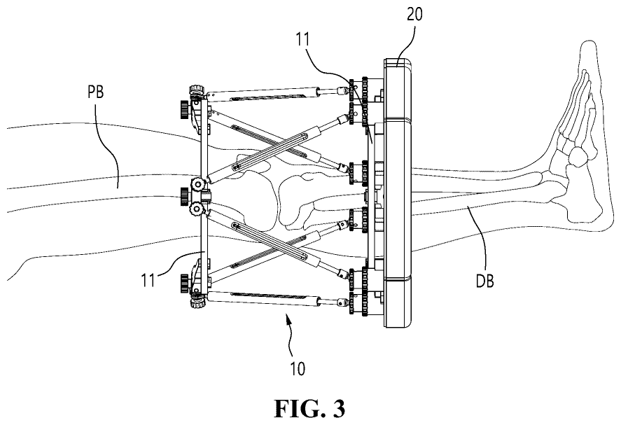

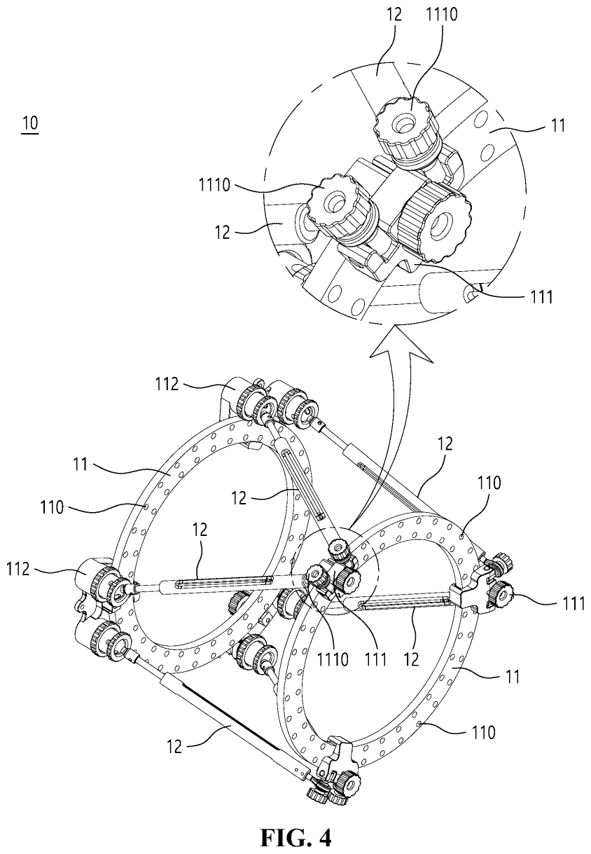

FIG. 1 is a perspective view showing a fracture reduction robot having a wireless driving part according to an embodiment of the present invention, FIG. 2A is an exploded perspective view showing a fracture reduction part and a driving part of FIG. 1, FIG. 2B is a perspective view showing a state in which the fracture reduction part and the driving part are coupled to each other, FIG. 3 is a side view showing a state in which the fracture reduction robot of FIG. 1 is fixed to a fractured area, and FIG. 4 is a perspective view showing the fracture reduction part of FIG. 2A.

Referring to FIG. 1, a fracture reduction robot 1 having a wireless driving part according to an embodiment of the present invention includes a fracture reduction part 10 and a driving part 20.

First, the fracture reduction part 10 is fixed directly to a fractured area of a patient in such a way as to pass the patient's arm or leg therethrough. The fracture reduction part 10 serves to fix and pull the fractured area and thus reduce a fractured bone.

Referring to FIG. 2A, the fracture reduction part 10 includes ring frames 11 and struts 12.

The ring frames 11 allow the patient's arm or leg to pass therethrough. The ring frames 11 are fixed to the fractured area after passing the patient's arm or leg therethrough.

The ring frames 11 have the shapes of rings, and otherwise, they have the shapes of ‘C’. The ring frames 11 may not be limited thereto.

In this case, each ring frame 11 has coupling holes 110 formed to pass therethrough at given intervals along a circumferential direction thereof. The coupling holes 110 are coupled to bone fixation frames (not shown) used for fracture reduction surgery.

In detail, each bone fixation frame (not shown) typically consists of a steel plate coming into contact with a bone, a ‘¬’-shaped bar built on the ring frame 11, and a bolt for coupling the steel plate and the bar, but the bone fixation frame may not be limited thereto.

Further, the ring frames 11 are provided to be spaced apart from each other by a given distance. The ring frames 11 are fixed to the fractured area in a longitudinal direction of the fractured area.

Desirably, two ring frames 11 are fixed to a distal bone (DB) and a proximal bone (PB), and one or more struts 12 are connected between the ring frames 11.

In this case, the distal bone (DB) refers to the bone distant from the heart when the fractured area is open, and the proximal bone (PB) refers to the bone close to the heart.

For example, as shown in FIG. 3, if the fractured area is the fibular, the distal bone (DB) refers to the bone close to the foot, and the proximal bone (PB) refers to the bone close to the thigh.

As shown in FIG. 4, the ring frames 11 are connected to the struts 12 by means of top connectors 111 and bottom connectors 112.

In this case, the top connectors 111 are located on the ring frame 11 connected to the distal bone (DB), and the bottom connectors 112 are located on the ring frame 11 connected to the proximal bone (PB). Since the fixed positions of the ring frames 11 are varied according to the installation direction of the fracture reduction part 10 on the fractured area, however, the connection positions of the top connectors 111 and the bottom connectors 112 may not be limited thereto.

The ring frames 11 fixed to the distal bone (DB) and the proximal bone (PB) perform the fracture reduction under the operations of the struts 12. An explanation of the operations of the struts 12 will be given in detail later.

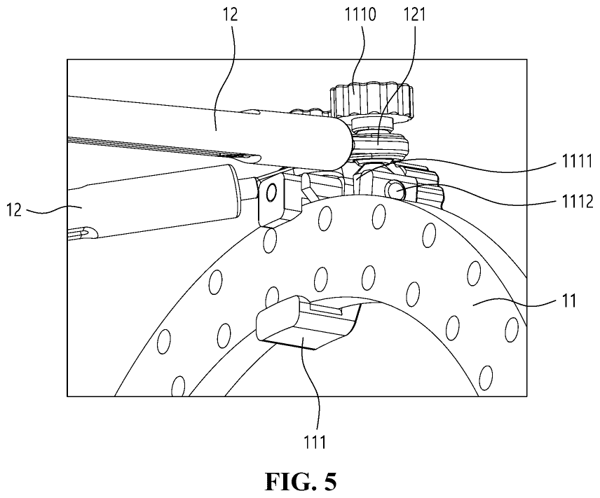

Hereinafter, the bottom connectors 112 and the top connectors 111 for fixing the ring frames 11 and the struts 12 to one another will be described in detail with reference to FIGS. 5 to 9.

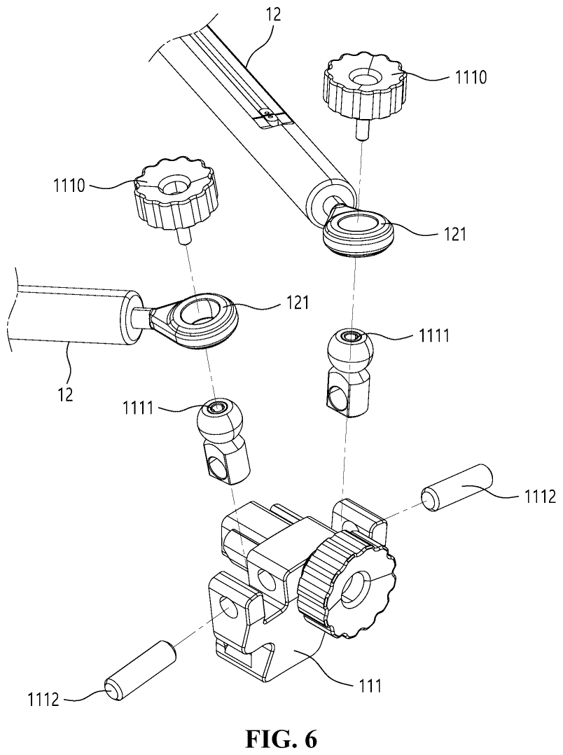

First, FIG. 5 is a perspective view showing a state where the top connectors 111 of FIG. 4 are connected to the ring frame 11, and FIG. 6 is an exploded perspective view showing the top connectors 111 and the struts 12 of FIG. 4.

Referring to FIG. 4, each top connector 111 connects one end of the struts 12 to the ring frame 11.

In detail, the top connector 111 has a clamp. The clamp is brought into close contact with the periphery of the ring frame 11 to prevent the top connector 111 from being varied in angle or prevent the top connector 111 from slipping along the periphery of the ring frame 11.

The top connector 111 has fixing grooves formed on top of the clamp. Fixing pins 1111 are inserted into the fixing grooves, and rotational pins 1112 are inserted into the fixing grooves and hollow portions of the fixing pins 1111.

Further, each fixing pin 1111 has a groove formed on top thereof in such a way as to fasten an escape prevention member 1110 thereto.

As shown in FIG. 5, the top connector 111 is connected to the struts 12. In this case, each strut 12 has a rod formed on one end thereof, and the rod has a through hole 121 formed in the middle portion thereof.

In more detail, the escape prevention member 1110 is inserted into the groove formed on top of the fixing pin 1111 and the through hole 121 of the strut 12, so that the top connector 111 and the strut 12 are connected to each other around the escape prevention member 1110 and the corresponding rotational pin 1112.

Desirably, the top connector 111 is connected to one pair of struts 12 through two escape prevention members 1110, and when the ring frames 11 and the struts 12 are coupled to one another by a user, the user's load can decrease.

Under the above-mentioned configuration, the top connector 111 fastens one or more struts 12 to the ring frame 11, and each strut 12 rotates around the through hole 121 and the rotational pin 1112. Further, the length of the strut 12 can be varied to allow the position and angle of the ring frame 11 to be changed.

An explanation of the change in length of the strut 12 will be given in detail later.

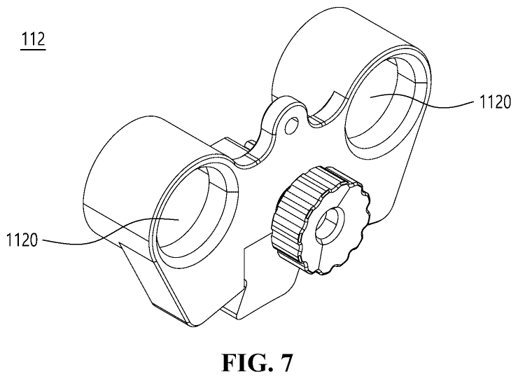

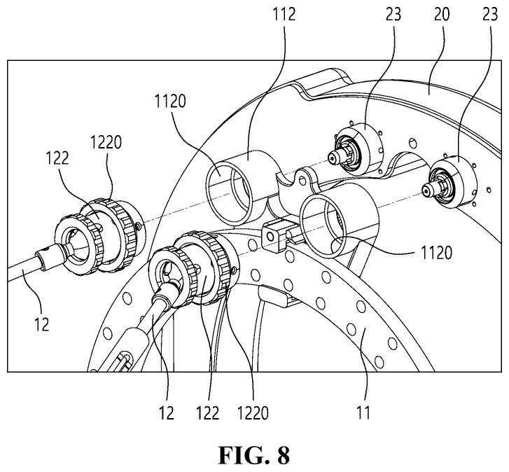

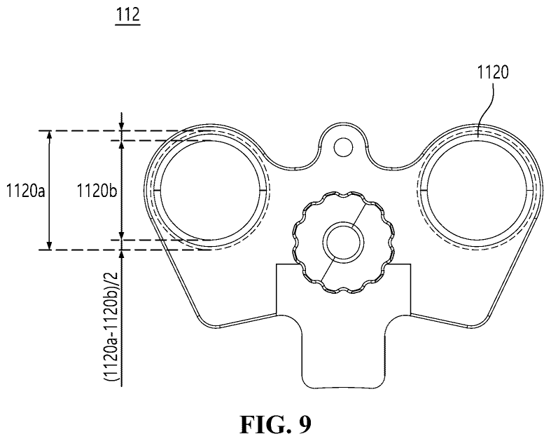

Further, FIG. 7 is a perspective view showing the bottom connectors 112 of FIG. 4, FIG. 8 is an exploded perspective view showing the bottom connectors 112 to which the ring frame 11 is connected, the struts 12, and the driving part 20 of FIG. 4, and FIG. 9 is a plan view showing a difference between diameters of first and second connection holes 1120 of the bottom connector 112 of FIG. 7.

Referring to FIGS. 7 and 8, the bottom connector 112 has a clamp. The clamp is brought into close contact with the periphery of the ring frame 11 to prevent the bottom connector 112 from being varied in angle or prevent the bottom connector 112 from slipping along the periphery of the ring frame 11.

Further, the bottom connector 112 connects the driving part 20 and the struts 12 to each other through connection holes 1120.

To do this, each connection hole 1120 has diameters corresponding to the driving part 20 and the strut 12.

In detail, the diameter of the connection hole 1120 coming into contact with the driving part 20 is equal to the size of a driving part coupler 23, and the diameter of the connection hole 1120 coming into contact with the strut 12 is equal to the size of a power transmission member 122.

In this case, the connection hole 1120 open toward the strut 12 is called a first connection hole 1120a, and the connection hole 1120 open toward the driving part 20 is called a second connection hole 1120b.

That is, as shown in FIG. 9, the bottom connector 112 has a difference (1120a-1120b/2) between the diameters of the first connection hole 1120a and the second connection hole 1120b.

Accordingly, the driving part coupler 23 and the power transmission member 122 of the strut 12 are received in the connection hole 1120 so that the connection therebetween is guided to allow the driving part 20 to be more easily mounted onto the fracture reduction part 10 by the user.

In this case, the power transmission member 122 of the strut 12 is located on the other end of the strut 12 to receive the operation of the driving part 20, and the driving part coupler 23 transmits the operation of the driving part 20. Explanations of the respective components will be given in detail below.

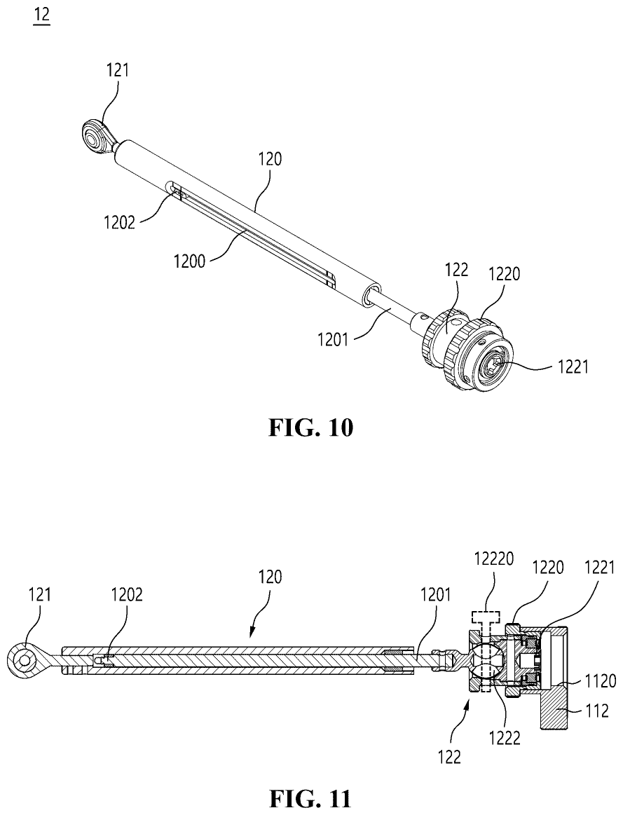

Further, FIG. 10 is a perspective view showing the strut 12 of FIG. 4, and FIG. 11 is a sectional view showing an interior of the strut 12 of FIG. 10 and a state where the bottom connector 112 is connected to the strut 12.

Referring to FIG. 10, the strut 12 is variable in length, like an actuator or cylinder, to allow the ring frames 11 to be changed in position. Each strut 12 includes a strut body 120, the through hole 121, and the power transmission member 122.

Further, the strut body 120 includes a sight gauge 1200, a cylinder 1201, and a measurer 1202.

The sight gauge 1200 is located on the periphery of the strut body 120 in a longitudinal direction of the strut body 120 and represents a maximum contraction length and a maximum extension length of the strut 12.

The cylinder 1201 is movable inside the strut body 120 to allow the entire length of the strut 12 to be varied. The cylinder 1201 moves in the longitudinal direction of the strut body 120 through the power received from the driving part 20.

As shown in FIG. 11, the measurer 1202 is located on the end of the cylinder 1201 and reciprocates inside the sight gauge 1200 in such a way as to be located at a position representing the length of the strut 12, thereby allowing the length of the strut 12 to be recognized with the naked eye of the user.

Without being necessarily limited to the above-mentioned physical method for measuring the length of the strut 12 with the naked eye of the user, of course, an electronic method for measuring the length of the strut 12 through the operation of the driving part 20 may be adopted together with the physical method as mentioned above.

As a result, the entire length of each strut 12 can be recognized by the user through the sight gauge 1200 and the measurer 1202, and further, the shape of the fracture reduction part 10 fixed to the fractured area is recognizable according to the lengths of the struts 12.

The through hole 121 is formed in the shape of a doughnut on one end of the strut body 120, and a bearing is located on the inner peripheral surface of the through hole 121 to provide easiness in rotation.

The through hole 121 allows the escape prevention member 1110 to pass therethrough and is thus connected to the top connector 111. That is, as shown in FIG. 6, the through hole 121 is fastened to the fixing pin 1111 through the escape prevention member 1110 of the top connector 111.

That is, the through hole 121 serves to allow the strut 12 to rotate on the top connector 111 around the escape prevention member 1110 and the fixing pin 1111.

The power transmission member 122 is located on the other end of the strut body 120 and thus connected to the bottom connector 112. That is, the power transmission member 122 is connected to the driving part 20 through the bottom connector 112 and receives the power from the driving part 20.

Referring to FIG. 11, the power transmission member 122 includes a pipe thread 1220, a key groove 1221, and a ball joint 1222.

The pipe thread 1220 is brought into contact with the bottom connector 112.

The pipe thread 1220 serves to couple the bottom connector 112 and the strut 12 to each other in such a way as to extend up to the inner peripheral surface of the bottom connector 112 and be thus brought into contact with the bottom connector 112.

The key groove 1221 is connected directly to the driving part 20 and thus receives the power of the driving part 20.

The ball joint 1222 inserts a pin 12220 passing through the power transmission member 122 from the outside of the power transmission member 122 thereinto, so that the ball joint 1222 rotates around the pin 12220.

Through the height-variable properties of the struts 12, the shapes of the struts 12 fastened to the corresponding top connector 111, and the power transmission members 122 of the struts 12, the struts 12 allow the ring frames 11 to perform linear axial movements, horizontal straight movements, and vertical straight movements, so that they apply six degrees of freedom to the ring frames 11 to allow the ring frames to perform pitch, yaw, and roll rotations.

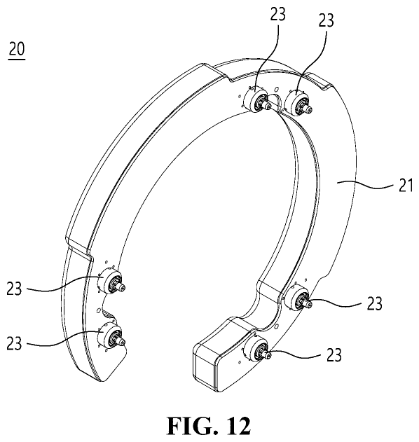

Hereinafter, a configuration and method for allowing the power of the driving part 20 to be transmitted to the struts 12 according to the embodiment of the present invention will be explained with reference to FIGS. 12 to 16.

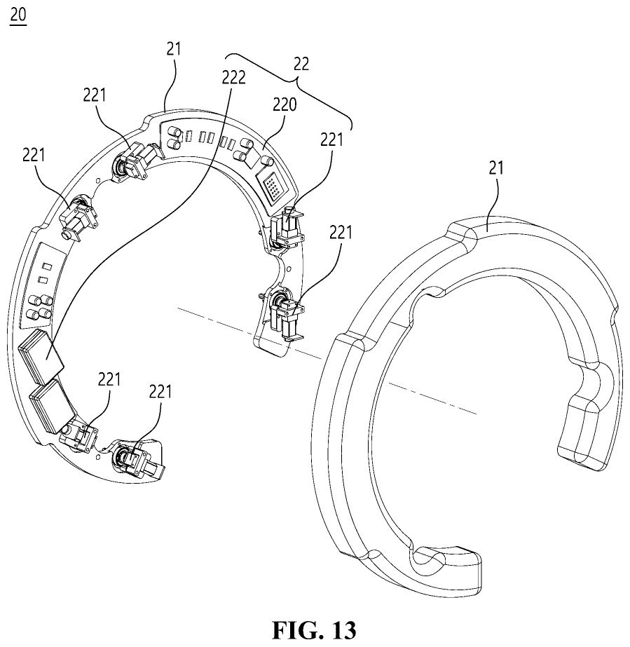

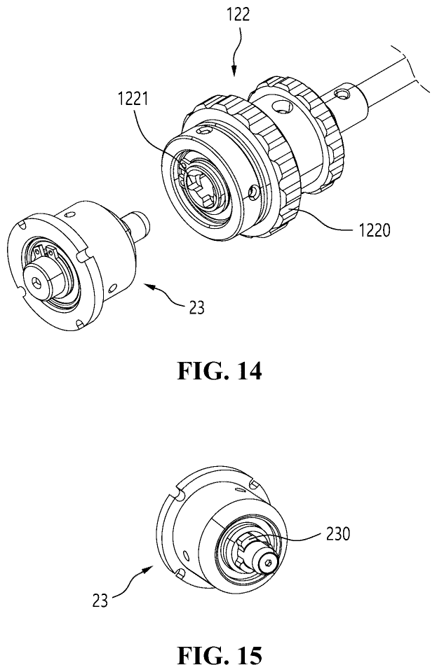

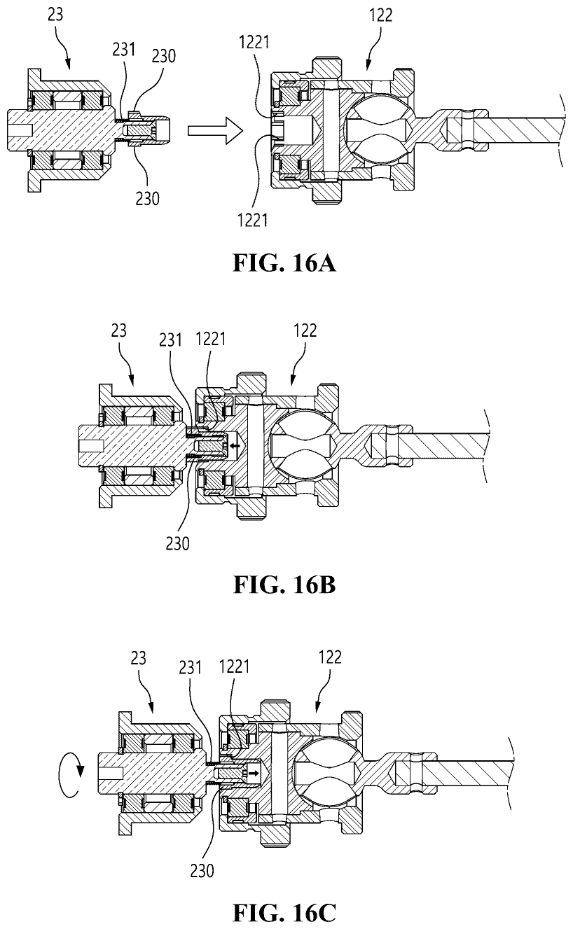

FIG. 12 is a perspective view showing the driving part of FIG. 2A, FIG. 13 is a perspective view showing the controller of the driving part of FIG. 12, FIG. 14 is a perspective view showing the driving part coupler of FIG. 2A and the power transmission member of the strut, FIG. 15 is a perspective view showing clutch teeth of the driving part coupler of FIG. 14, FIG. 16A is a sectional view showing a state before the driving part coupler of FIG. 14 is connected to a connection portion of the strut, FIG. 16B is a sectional view showing a state in which the driving part coupler of FIG. 14 is inserted into the connection portion of the strut, and FIG. 16C is a sectional view showing a state in which the driving part coupler of FIG. 14 is connected to the connection portion of the strut.

Referring to FIG. 12, the driving part 20 has the driving part couplers 23 protruding outward therefrom in such a way as to be connected to the ring frame 11. The driving part 20 transmits the power to the struts 12 through the driving part couplers 23, and in this case, the driving part 20 is a wireless driving part without a communication wire and a power wire.

Further, referring to FIG. 2B, driving part-fixing pins P are provided between the driving part 20 and the ring frame 11 to fasten them to each other, and after the driving part-fixing pins P pass through the driving part 20, one end of each pin P is fixed to the ring frame 11, so that the driving part-fixing pins P generate forces for fixing the driving part 20 and the ring frame 11 to each other.

Referring to FIG. 13, the driving part 20 includes a driving part housing 21 and a controller 22.

First, the driving part housing 21 has the shape of a ring through which the patient's arm or leg passes. In this case, the driving part housing 21 has an inner diameter longer than an outer diameter of the ring frame 11.

That is, the driving part housing 21 is bigger than the ring frame 11 to cause no interference with the movement of the ring frame 11 when the patient's arm or leg passes through the ring frame 11.

Desirably, as shown in FIG. 13, the driving part housing 21 is partially incised so that the fracture reduction part 10 and the driving part 20 can be easily coupled to each other by inserting the user's hand into the incised portion.

Further, the driving part housing 21 has holes through which the driving part couplers 23 mounted on driving motors 221 of the controller 22 pass.

The controller 22 is located inside the driving part housing 21 and serves to control the struts 12.

Components of the controller 22 are located on one side of the interior of the driving part housing 21. The controller 22 includes a main controller 220, the driving motors 221, and a power source part 222.

The main controller 220 serves to control the operation of the fracture reduction part 10.

In detail, the main controller 220 performs radio communication and controls the driving motors 221 and the power source part 222 according to information after the radio communication to allow the fracture reduction part 10 to reduce the fractured area.

The main controller 220 controls revolutions per minute and directions of the driving motors 221 to allow the lengths of the struts 12 to be varied from the current positions of the struts 12 to which the fracture reduction part 10 is fixed. For example, the main controller 220 allows the struts 12 to be varied in length so that the ring frames 11 are changed in angle and position.

Further, the main controller 220 performs communication with other terminals through a pairing method using direct contact, such as near field communication (NFC), and a long distance wireless communication method using an industrial, scientific and medical (ISM) band.

The number of driving motors 221 corresponds to the number of struts 12, and the driving motors 221 produce power for varying the lengths and directions of the struts 12 under the control of the main controller 220.

Two driving motors 221 as one pair are located in three directions according to the positions of the struts 12, and three pairs of driving motors 221 transmit the power to the respective struts 12 through the driving part couplers 23. However, the positions of the driving motors 221 may not be limited thereto.

Further, the main controller 220 monitors the revolutions per minute and directions of the driving motors 221 corresponding to the respective struts 12 and thus calculates the current lengths of the struts 12.

The power source part 222 is provided in the form of a battery to apply a power source to the main controller 220 and the driving motors 221 and charged wirelessly. The power source part 222 is charged by means of magnetic induction or resonant reduction, but it may not be limited thereto.

The driving part couplers 23 are located on one surface of the driving part housing 21 in such a way as to be connected to the struts 12.

The driving part couplers 23 are located on the positions where the driving motors 221 are located and transmit the power of the driving motors 221 to the struts 12.

In this case, the driving part couplers 23 are connected to the struts 12 through the bottom connectors 112.

Referring to FIGS. 14 and 15, each driving part coupler 23 includes the clutch teeth 230.

The clutch teeth 230 are engaged with the key groove 1221 of the strut 12.

In detail, as shown in FIG. 14, one or more clutch teeth 230 are formed in a circumferential direction of the driving part coupler 23, but they are exemplary. Therefore, they may have various shapes detachably mounted onto the key groove 1221.

Desirably, the clutch teeth 230 are spaced apart from one another by a given distance in the circumferential direction of the driving part coupler 23.

Further, as shown in FIG. 16A, the driving part coupler 23 has an elastic member 231 connected to the clutch teeth 230.

The clutch teeth 230 are pushed toward the key groove 1221 from the driving part coupler 23 or pulled in the opposite direction to the pushed direction by means of the elastic member 231 according to the attachment and detachment to and from the key groove 1221.

The elastic member 231 may include all types of members capable of pushing the clutch teeth 230 toward the key groove 1221, such as a coil spring, a leaf spring, a disc spring, and the like, but it may not be limited thereto.

Desirably, the elastic member 231 is a coil spring allowing the ends of the clutch teeth 230 to pass therethrough.

That is, as shown in FIG. 16A, the driving part coupler 23 faces the connected surface of the power transmission member 122, and if it is desired to connect the strut 12 and the driving part coupler 23 to each other, as shown in FIG. 16B, a state in which the engaged coupling between the clutch teeth 230 and the key groove 1221 is delayed through the elastic force of the elastic member 231 is kept. Further, as shown in FIG. 16C, the rotational force of the corresponding driving motor 221 is added, so that the clutch teeth 230 and the key groove 1221 are engagedly coupled to each other.

As a result, the plurality of struts 12 and the plurality of driving part couplers 23 are mounted at a time on the driving part 20, so that the coupling between the fracture reduction part 10 and the driving part 20 can be more conveniently performed.

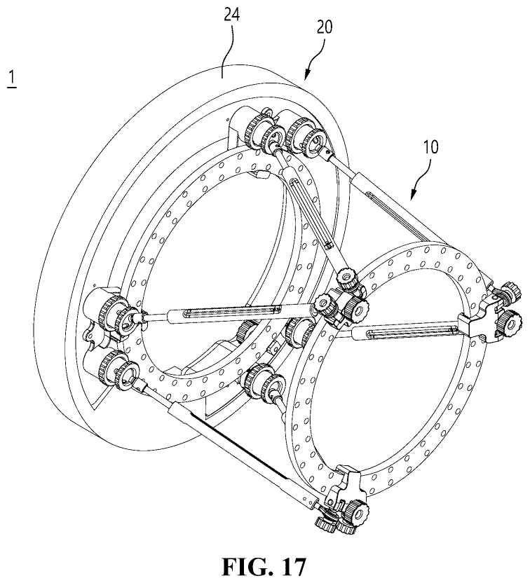

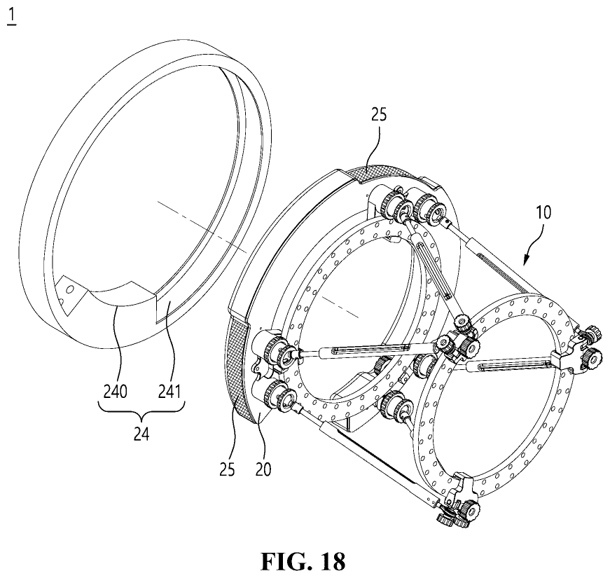

FIG. 17 is a perspective view showing a fracture reduction robot having a wireless driving part according to another embodiment of the present invention, and FIG. 18 is an exploded perspective view showing the driving part and a cooler of FIG. 17.

Referring to FIG. 17, a driving part 20 of a fracture reduction robot according to another embodiment of the present invention further includes a cooler 24 and mesh screens 25.

In this case, the fracture reduction robot 1 having the wireless driving part according to another embodiment of the present invention is substantially the same as the fracture reduction robot 1 according to one embodiment of the present invention, excepting that the driving part 20 further includes the cooler 24 and the mesh screens 25.

Therefore, an explanation of the cooler 24 and the mesh screens 25 will be given below.

Referring to FIGS. 17 and 18, the cooler 24 serves to lower an amount of heat generated from the driving part 20. To do this, the cooler 24 includes a temperature converter 240 and a heat moving space portion 241.

The temperature converter 240 converts hot air generated from the driving part 20 into cold air.

In more detail, the temperature converter 240 basically includes an air blower for providing a flow of air through which internal heat is cooled and a heat pump such as a heat conversion device or thermoelectric device (Peltier) using a fluid for providing improved cooling performance, but the temperature converter 240 may not be limited thereto.

In this case, the hot air is the heat conducted along the outer peripheral surface of the driving part 20 or the heat circulated along the heat moving space portion 241 after emitted from the mesh screens 25.

Further, the temperature converter 240 is open in the opposite direction to a direction to which the fractured area is fixed, so that the hot air is emitted through the open portion, but the temperature converter 240 may not be limited thereto.

As shown in FIG. 18, the temperature converter 240 has one or more holes formed on the sides toward the heat moving space portion 241 in such a way as to allow the hot air to be introduced thereinto, and after the hot air has been converted into the cold air, the temperature converter 240 emits the cold air toward the heat moving space portion 241.

The heat moving space portion 241 is formed along the peripheral surface of the driving part 20. The heat moving space portion 241 has connection protrusions (not shown) protruding from a portion coming into contact with the driving part 20 so that it is not separated from the driving part 20.

Further, the heat moving space portion 241 is formed of an elastic material and thus fixedly surrounds the outer peripheral surface of the driving part 20.

The heat moving space portion 241 may be formed of all types of materials, such as rubber, vinyl, spandex, and the like, that surround the driving part 20 to provide a path along which the cold air of the temperature converter 240 moves, but the heat moving space portion 241 may not be limited thereto.

Further, the heat moving space portion 241 has a moisture absorbing material along the inner peripheral surface thereof. The moisture absorbing material prevents condensation from occurring due to the movements of the hot air emitted from the mesh screens 25 and the cold air generated from the temperature converter 240.

The moisture absorbing material includes all types of materials capable of absorbing moisture or dews, such as paper, zeolite, charcoal, nylon, ABS resin, polycarbonate, polymer resin, and the like, but the moisture absorbing material may not be limited thereto.

Further, the mesh screens 25 are located on the side peripheral surfaces of the driving part 20 and thus transmit the cold air of the cooler 24 to the driving motors 221.

In detail, the mesh screens 25 allow the hot air generated from the driving motors 221 with the largest amount of heat in the controller 22 to be emitted toward the heat moving space portion 241 located within the shortest distance from the corresponding driving motors 221.

After that, the mesh screens 25 pass the cold air emitted toward the heat moving space portion 241 therethrough and thus directly transmit the cold air to the driving motors 221 and the main controller 220.

Further, the mesh screens 25 have fine mesh sizes allowing only gas to pass therethrough so that dust, which is introduced into the open portion of the temperature converter 240, is prevented from entering the driving part housing 21.

That is, the mesh screens 25 easily take the heat out of the driving motors 221 and the main controller 220 and thus transmit the heat to the temperature converter 240, and further, the mesh screens 25 directly transmit the cold air converted from the temperature converter 240 to the driving motors 221 and thus decrease the amount of heat generated from the driving part 20 more effectively.

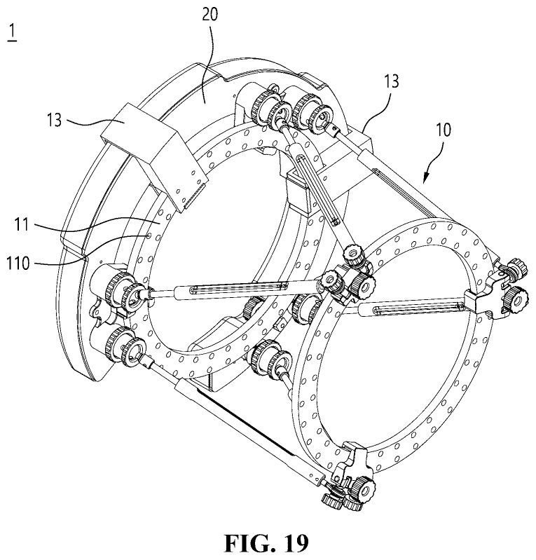

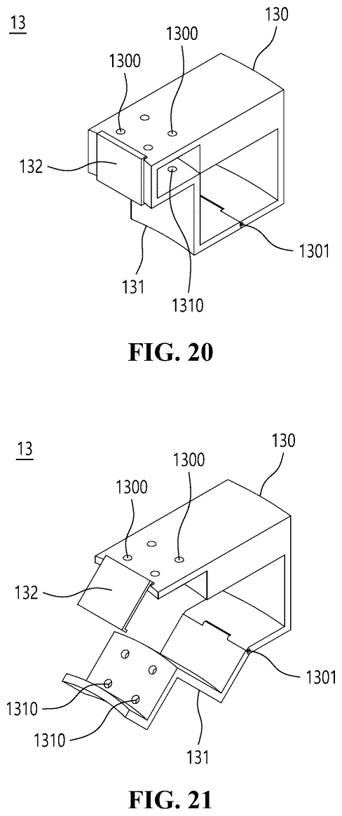

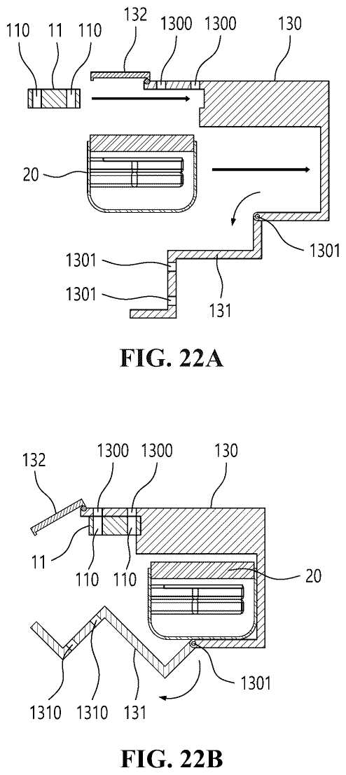

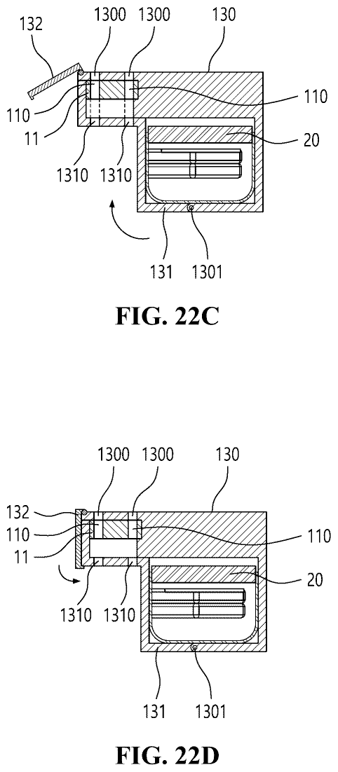

FIG. 19 is a perspective view showing a fracture reduction robot having a wireless driving part according to yet another embodiment of the present invention, FIG. 20 is a perspective view showing an auxiliary connector of FIG. 19, FIG. 21 is a perspective view showing a state where the auxiliary connector of FIG. 20 is open, FIG. 22A is an exemplary view showing a state before the auxiliary connector of FIG. 19 is connected to the ring frame and the driving part, FIG. 22B is an exemplary view showing a state in which a top portion of the auxiliary connector of FIG. 19 is seated onto tops of the ring frame and the driving part, FIG. 22C is an exemplary view showing a state in which an underside portion of the auxiliary connector of FIG. 19 rotates to surround the ring frame and the driving part, and FIG. 22D is an exemplary view showing a state in which a closer of the auxiliary connector of FIG. 19 fixes the top portion and the underside portion thereto.

Referring to FIG. 19, a fracture reduction part 10 of a fracture reduction robot according to yet another embodiment of the present invention further includes auxiliary connectors 13.

In this case, the fracture reduction robot 1 having the wireless driving part according to yet another embodiment of the present invention is substantially the same as the fracture reduction robot 1 according to one embodiment of the present invention, excepting that the fracture reduction part 10 further includes the auxiliary connectors 13.

Therefore, an explanation of the auxiliary connectors 13 will be given below.

Referring to FIG. 20, each auxiliary connector 13 surrounds the ring frame 11 and the driving part 20 to help them connected to each other and includes a top portion 130, an underside portion 131, and a closer 132.

The top portion 130 is located above the ring frame 11 and the driving part 20 to surround them and has a plurality of first through holes 1300 formed on top thereof.

In this case, the positions and intervals of the first through holes 1300 are the same as of the coupling holes 110 of the ring frame 11, so that when the fractured area is fixed to the ring frame 11, some of the coupling holes 110 are not covered with the top portion 130.

In this case, the top portion 130 allows a portion in which the ring frame 11 and the driving part 20 are spaced apart from each other to be flat and has the same side peripheries as the ring frame 11 and the driving part 20.

Under such a configuration, the top portion 130 allows the ring frame 11 and the driving part 20 to be coupled to each other, without any gap.

Further, the top portion 130 extends downward to surround the underside of the driving part 20 in such a way as to be connected to the underside portion 131 through a rotational shaft 1301.

The underside portion 131 is located under the top portion 130 and rotates around the rotational shaft 1301 to surround the undersides of the ring frame 11 and the driving part 20.

As shown in FIG. 21, the underside portion 131 has one or more second through holes 1310 formed on top thereof in such a way as to vertically correspond to the first through holes 1300. In this case, the second through holes 1310 serve to insert the bone fixing frames for fixing the fractured area through the ring frame 11, in the same manner as the first through holes 1300.

The underside portion 131 is formed correspondingly to the outer peripheral surfaces of the ring frame 11 and the driving part 10, in the same manner as the top portion 130, and thus surrounds the ring frame 11 and the driving part 20, without any gap. Unlike the top portion 130, the underside portion 131 is provided in the form of stairs, but it may not be limited thereto.

The closer 132 serves to fix the top portion 130 to the underside portion 131.

In detail, the closer 132 is located on the end of the top portion 130. If the underside portion 131 rotates and thus comes into close contact with the top portion 130, the closer 132 rotates around the top of the top portion 130 and is thus fixed to the underside portion 131.

The closer 132 has a protrusion on a position coming into contact with the underside portion 131, and the underside portion 131 has a groove on the same position as the protrusion. As a result, the closer 132 is fittedly coupled to the underside portion 131 to prevent the ring frame 11 and the driving part 20 connected through the auxiliary connector 13 from being separated from each other.

Under such a configuration, as shown in FIGS. 22A and 22B, the top portion 130 is seated on top of the ring frame 11 and the driving part 20. After that, as shown in FIG. 22C, the underside portion 131 rotates around the rotational shaft 1301 and comes into close contact with the top portion 130.

Lastly, as shown in FIG. 22D, the closer 132 fixes the top portion 130 to the underside portion 131, so that during the fracture reduction surgery, the auxiliary connectors 13 can prevent the ring frame 11 and the driving part 20 from being separated from each other.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above teachings.

### EXPLANATIONS OF REFERENCE NUMERALS

1: Fracture reduction robot
10: Fracture reduction part
11: Ring frame
110: Coupling hole
111: Top connector
1110: Escape prevention member
1111: Fixing pin
1112: Rotational pin
112: Bottom connector
1120: Connection hole
1120a: First connection hole
1120b: Second connection hole
12: Strut
120: Strut body
1200: Sight gauge
1201: Cylinder
1202: Measurer
121: Through hole
122: Power transmission member
1220: Pipe thread
1221: Key groove
1222: Ball joint
12220: Pin
13: Auxiliary connector
130: Top portion
1300: First through hole
1301: Rotational shaft
131: Underside portion
1310: Second through hole
132: Closer
20: Driving part
21: Driving part housing
22: Controller
220: Main controller
221: Driving motor
222: Power source part
23: Driving part coupler
230: Clutch teeth
231: Elastic member
24: Cooler
240: Temperature converter
241: Heat moving space portion
25: Mesh screen
PB: Proximal bone
DB: Distal bone
P: Driving part fixing pin

1. A fracture reduction robot comprising:
a fracture reduction part having a plurality of ring frames for surrounding a fractured area of a patient and one or more struts disposed between the plurality of ring frames to adjust a distance between the plurality of ring frames; and
a wireless driving part connected to any one of the plurality of ring frames to transmit power for adjusting the distance between the plurality of ring frames.

2. The fracture reduction robot of claim 1, wherein the fracture reduction part further comprises:
one or more top connectors, wherein the one or more top connectors are connected to one end of the one or more struts; and
one or more bottom connectors for connecting an other end of the one or more struts to the driving part.

3. The fracture reduction robot of claim 2, wherein each of the one or more struts comprises:
a strut body variable in length and having a sight gauge disposed in a longitudinal direction thereof and a measurer configured to move in an interior of the sight gauge according to the variation in length of the strut body to measure a length of the strut;
a through hole formed on one end of the strut body and configured to be fastened to the a corresponding top connector; and
a power transmission member disposed on an other end of the strut body in and connected to the-a corresponding bottom connector to receive the power from the driving part for adjusting the length of the strut body.

4. The fracture reduction robot of claim 3, wherein each of the one or more top connectors comprises:
one or more fixing pins passing through one end of corresponding strut bodies to allow the struts to be fixed to the top connector;
one or more escape prevention members coupled to the corresponding one or more fixing pins to prevent the struts fixed to the top connector from escaping therefrom; and
one or more rotational pins passing through one side peripheries of the corresponding one or more fixing pins from an outside thereof to adjust an angle of the fixing pins,
wherein the one or more struts are variable in angle around the corresponding one or more rotational pins of the one or more top connectors.

5. The fracture reduction robot of claim 3, wherein the driving part comprises:
a driving part housing, wherein a limb of a patient is configured to pass through the driving part housing;
a controller disposed inside the driving part housing to provide the power to the one or more struts; and
one or more driving part couplers disposed on one surface of the driving part housing and connected to the power transmission members of the one or more struts through the one or more bottom connectors to transmit the power to the one or more struts to adjust the lengths of the strut bodies.

6. The fracture reduction robot of claim 5, wherein each of the one or more bottom connectors comprises:
one or more first connection holes facing the corresponding struts and having a diameters equal to a diameters of the power transmission members of the struts; and
one or more second connection holes facing the corresponding driving part couplers and having a diameters equal to a diameters of the driving part couplers,
wherein the bottom connectors connect the power transmission members and the driving part couplers to each other.

7. The fracture reduction robot of claim 5, wherein each power transmission member comprises:
a pipe thread for coupling the corresponding strut to the corresponding bottom connector;
a key groove configured to engage with the corresponding driving part coupler; and
a ball joint having a pin passing therethrough from an outside thereof to rotate around the pin.

8. The fracture reduction robot of claim 5, wherein the controller comprises:
a main controller for controlling an operation of the fracture reduction part;
one or more driving motors disposed to correspond to the one or more struts to vary the lengths and directions of the one or more struts under the control of the main controller; and
a power source configured to apply power to the main controller and the one or more driving motors.

9. The fracture reduction robot of claim 7, wherein each driving part coupler comprises clutch teeth engaged with the key groove of a corresponding power transmission member and an elastic member for pushing the clutch teeth toward the key groove,
wherein the clutch teeth are engaged with the key groove through an operation of the controller.

10. The fracture reduction robot of claim 78, wherein the driving part further comprises:
a cooler for lowering an amount of heat generated from the controller; and
mesh screens disposed on side peripheral surfaces of the driving part where the one or more driving motors are located to allow heat generated from the one or more driving motors to be emitted to an outside,
wherein cold air is transmitted directly or indirectly to the one or more driving motors by means of the mesh screens.

11. The fracture reduction robot of claim 1, wherein the fracture reduction part further comprises one or more auxiliary connectors for surrounding a ring frame close to the driving part and the driving part to fix the driving part to the close ring frame.
